(19) United States
(12) Patent Application Publication
(10) Pub. No.: US 2009/0024044 A1
(43) Pub. Date: Jan. 22, 2009

(54) DATA RECORDING FOR PATIENT STATUS ANALYSIS

(75) Inventors: Juha Virtanen, Helsinki (FI); Seppo Iikka Juhani Virtanen, Espoo (FI)

Correspondence Address: ANDRUS, SCEALES, STARKE & SAWALL, LLP, 100 EAST WISCONSIN AVENUE, SUITE 1100, MILWAUKEE, WI, 53202, US

(73) Assignee: THE GENERAL ELECTRIC COMPANY, Schenectady, NY (US)

(21) Appl. No.: 11/778,722

(22) Filed: Jul. 17, 2007

(51) Int. Cl. A61B 5/0476

(52) U.S. Cl. 600/509; 600/544; 600/547

(57) ABSTRACT

A method and system for analyzing the status of a subject. A cordless recording device comprising a set of electrodes is attached on the forehead of the subject to collect brain wave signal data from the subject. Based on the brain wave signal data, status information indicative of the status of the subject during a measurement period is generated. A bioimpedance signal is further measured through two electrodes of the electrode set and heart rate data is derived from the bioimpedance signal. The heart rate data is utilized to enhance the quality of the status information, which is typically sleep state information. The invention also relates to a recording device attachable to the forehead of the subject. The recording may comprise a data memory for storing the data measured from the subject during the measurement period.

### FIELD OF THE INVENTION

The present invention relates generally to the recording of physiological data for analysis of patient status. The invention is particularly suitable for sleep analyses.

### BACKGROUND OF THE INVENTION

Sleep recordings are important for the analysis, diagnosis, and treatment of various sleep disorders. Sleep staging, in turn, is a vital step in sleep analysis. Sleep staging is normally performed using the traditional Rechtschaffen & Kales (R&K) rules, which classify sleep into six separate stages: wake, rapid eye movement (REM) sleep, and S1 (light sleep) to S4 (deep sleep). Also indices like number of arousals or micro-arousals, total sleeping time, duration of movement time, or sleep latency can be used to characterize the sleep quality.

One drawback related to the traditional sleep studies is that the sleep recordings are made in separate sleep research laboratories. Due to the costly equipment involved and the trained personnel needed, the number of the laboratories is low and patients referred to a laboratory may have to travel over long distances. Furthermore, even though the sleep research laboratories may be comfortably furnished, many patients may find it hard to sleep naturally in these test environments, which detracts from the fidelity of the measurement data.

Various ambulatory devices have been developed to overcome this problem and to allow a user to carry out sleep recordings at home. Although recordings made at home provide more realistic information about the quality of sleep, these portable devices are often rather bulky and difficult to use, due to the rather high number of electrodes and connection wires needed. Furthermore, the user needs profound instructions for attaching the different electrodes at their respective measurement sites.

Smaller and more user-friendly devices are also available for sleep recordings. However, a major drawback related to many of these devices is that the recorded signal is not a brain wave signal but another signal that provides less relevant information about the sleep and thus makes the sleep analysis less accurate. Such a light-weight sleep recording device may be, for example, a sleep actigraph that contains an accelerometer for measuring patient movements during sleep. When sleeping, the patient wears a sleep actigraph around the wrist, for example, whereby the actigraph records the movements of the arm during the sleep. The movement data is then analyzed to evaluate the quality of sleep.

Portable recording devices have also been proposed, which rest on an EEG measurement and enable easy recording at home, and which are also rather non-noticeable and hence improve the diagnostic value of the data. U.S. Patent Application US 2006/0007796 discloses a recording device that comprises one or more electrodes/sensors and an electronic circuitry, including a memory, which are placed on an adhesive tape. The recording device suits particularly well for studying sleeping disorders, since the patient may easily attach the adhesive tape onto his or her forehead for collecting EEG data during sleep.

When comprehensive sleep recordings are carried out, heart rate (HR) is one of the parameters that are typically measured. Heart rate conveys information about the level of physical activity and metabolic rate. It is also well known in physiology that heart rate and temperature reflect the state of the autonomic nervous system (ANS) of the patient. Instantaneous heart rate values alone are hard to interpret unambiguously, but long-term HR trends correlated with other information, such as environmental events or physiological signals like EEG, are useful in determining the status of the patient.

From sleep medicine it is further well known that the activity of the autonomic nervous system is lowest during deep sleep, also known as slow wave sleep (SWS). Correspondingly, the activity is highest during REM (rapid eye movement) sleep. In sleep studies, heart rate trend is typically examined in parallel with other parameters of a polysomnographic recording. For example, abnormally elevated HR in the evening may be indicative of high stress. HR trend can also be used as additional information in sleeps stage scoring. For example, episodes of HR surges are likely to be REM sleep periods.

Heart rhythm also involves diagnostic value. Certain sleep stages are likely to elicit cardiac arrhythmias, cf. Principles and Practice of Sleep Medicine, Kryger, Roth & Dement, Sounders Company 2000; p. 187. Furthermore, obstructive sleep apnea is known to cause distinctive patterns in heart rate trend, cf. McNames & Fraser, Obstructive Sleep Apnea Classification based on Spectrogram Patterns in Cardiogram, Computers in Cardiology, 2000 Vol 27, 749-752.

A standard way to measure HR is to record ECG over patient's thorax. The electric fields produced by human heart extend also to the forehead and can be observed when the amplitude of electric brain activity is particularly low, for example in extremely deep anesthesia. However, the electrical brain activity of a normal healthy person is in practice at least ten times higher than the ECG on the forehead, cf. Bioelectromagnetism, Principles and Applications of Bioelectric and Biomagnetic Fields, Jaakko Malmivuo & Robert Plonsey, Oxford University Press 1995. Hence, the ECG is completely hidden under the EEG and cannot be used for reliable HR determination.

Consequently, light-weight and user-friendly recording devices, such as the one described in the above-mentioned U.S. Patent Application US 2006/0007796, are to be attached on the chest of the patient if the ECG is to be measured, i.e. the additional information provided by heart rate data, which would be desirable in sleep analysis, cannot be recorded by the device as the measurement is made from the forehead to obtain EEG signal data for sleep analysis.

It is also known that pulsating blood flow in aorta causes minor head and body movements. A ballistocardiogram recorded under optimal conditions conveys information not only about heart rate but also about blood volume pumped by the heart, called stroke volume. Same kind of signals can be recorded using an accelerometer placed on patient's forehead. Unfortunately, an acceleration signal from the forehead is not a reliable indicator of heart rate. This is because the head movement due to cardiac activity depends on numerous factors, including stroke volume, head support, posture etc.

One way to obtain reliable heart rate information from an instrument on patient's forehead is to measure light reflectance and/or absorption of skin. In this method patient's skin is illuminated using a red light source and the amount of light traveling through the skin is measured through a receiver. The absorption of light varies as a function of blood volume in the tissue. Hence, the obtained photoplethysmographic wave conveys information about the heart rate. If more than one wavelength is used, light reflectance and/or absorption may be used to obtain information about the oxygen saturation of arterial blood.

The above-described optical method provides reliable HR information, but has some technical drawbacks. First of all, it requires at least one optical transmitter and one receiver in contact with the skin. These additional sensor elements increase the complexity of the device and detract from its cost-effectiveness. In addition, the power consumption of the illumination of tissue is relatively high, which translates to increased battery size or shorter operating time.

The present invention seeks to alleviate or eliminate the above-mentioned drawbacks and to accomplish a mechanism that allows collection of both brain wave and heart rate data in small and light-weight battery powered devices attachable to patient's forehead.

### SUMMARY OF THE INVENTION

The present disclosure seeks to provide a novel mechanism that enables a light-weight data logger attachable to the forehead to collect both brain wave and heart rate data from a patient. The present disclosure further seeks to provide an arrangement that enables efficient analysis of the patient status based on multiple physiological parameters although the physiological data is collected through a user-friendly recording device attachable to the forehead without any connection wires.

In the present disclosure, brain wave signal data, typically EEG signal data is acquired from a patient through a cordless recording device attachable to the forehead of the patient and provided with an electrode set and an associated electronic circuitry including a data memory for storing the brain wave signal data measured from the patient. It is to be noted here that although the biopotential signal acquired from the subject is in this context termed brain wave signal data, it may also include other signal components, such as electromyographic (EMG) components caused by facial muscle activity. The term forehead here refers generally to the non-hairy area of the head from which brain wave signal data may be measured. This area includes not only the actual forehead, but also the temples and mastoids.

When the device is returned for analysis, status information indicative of the status of the patient during the measurement is generated based on the stored brain wave signal data. The status information typically indicates sleep states or stages during the measurement. The status information may also include various indices indicative of different sleep characteristics, like the number of arousals or micro-arousals, total sleeping time, duration of movement time, or sleep latency, which may be used to characterize sleep quality.

The electronic circuitry of the recording device further includes components for measuring bioimpedance signal data through two electrodes of the set. The pulsating blood volume component of the measured bioimpedance signal is employed to deduce heart rate information from the bioimpedance signal. The heart rate information may then be utilized to enhance the quality of the status information provided to the user. Quality enhancement here refers to improved information content from the point of view of the user.

The brain wave and HR data obtained enable an efficient off-line analysis when the recording device is returned to the doctor who has prescribed the examination. The heart rate information may be stored together with the brain wave signal data into the data memory of the recording device, but the recording device may also store the bioimpedance signal data from which the heart rate information may be deduced after the collected data is returned for off-line analysis. In a further embodiment of the invention, the data measured from the patient is transmitted wirelessly to an external analysis device, which may analyze the data off-line or on-line. Therefore, the recording device does not necessarily comprise the data memory but may transfer the data to the external device in an on-line manner.

Thus one aspect of the disclosure is providing a method for analyzing the status of a subject. The method comprises providing a cordless recording device comprising a set of electrodes and attaching the cordless recording device on the forehead of the subject. The method also includes collecting brain wave signal data from the subject through at least one electrode of the set, measuring bioimpedance through two electrodes of the set, thereby to obtain an impedance signal indicative of the bioimpedance of a signal path connecting the two electrodes, wherein the collecting and the measuring are performed over a measurement period and deriving heart rate data from the impedance signal, the heart rate data being indicative of the heart rate of the subject. The method further includes generating, based on the brain wave signal data, status information indicative of the status of the subject during the measurement period and utilizing the heart rate data to enhance the quality of the status information.

Another aspect of the disclosure is that of providing a system for analyzing the status of a subject. The system comprises a data collection unit configured to collect brain wave signal data from a subject through at least one electrode of a set of electrodes of a cordless recording device attachable to the forehead of the subject and an impedance measurement unit configured to measure bioimpedance through two electrodes of the set, thereby to obtain an impedance signal indicative of the bioimpedance of a signal path connecting the two electrodes. The system further comprises a heart rate analysis unit configured to derive heart rate data from the impedance signal, the heart rate data being indicative of the heart rate of the subject, a status analysis unit configured to generate, based on the brain wave signal data, status information indicative of the status of the subject during the measurement period, and a quality enhancing unit configured to enhance the quality of the status information based on the heart rate data.

The disclosure enables a user-friendly data recording process for acquiring the physiological data. Furthermore, the recording may be made with a device that is rather uncomplicated and thus also cost-effective for obtaining both brain wave and heart rate data from the patient. Furthermore, the power consumption of a signal generator required for the bioimpedance measurement is substantially lower than that of an optical transmitter-receiver pair, for example, required in the above-described optical method for acquiring heart rate data from the forehead.

A further aspect of the disclosure is that of providing a cordless recording device for recording physiological data from a subject. The cordless recording device comprises a set of electrodes and a first data collection unit configured to collect brain wave signal data from a subject through at least one electrode of the set. The device further comprises an impedance measurement unit configured to measure bioimpedance through two electrodes of the set, thereby to obtain an impedance signal indicative of the bioimpedance of a signal path connecting the two electrodes and a second data collection unit configured to collect pulse data indicative of the heart rate of the subject.

Depending on the embodiment, the cordless recording device may or may not be provided with a data memory for storing the brain wave signal data and the pulse data.

Other features and advantages of the disclosure will become apparent by reference to the following detailed description and accompanying drawings

### BRIEF DESCRIPTION OF THE DRAWINGS

In the following, the preferred embodiments are described more closely with reference to the examples shown in FIG. 1 to 9 in the appended drawings, wherein:

FIG. 1 illustrates one embodiment of the recording device and the data recording process;

FIG. 2 illustrates one embodiment of the recording device;

FIGS. 3a to 3c illustrate the measured bioimpedance signal in different points of the impedance measurement branch of the apparatus of FIG. 2;

FIG. 4 illustrates one embodiment of the overall analysis of the status of a patient;

FIG. 5 illustrates one embodiment of the sleep analysis;

FIGS. 6a and 6b show an example of the graphical presentation of the results of the sleep analysis;

FIG. 7 illustrates a further embodiment of the cordless recording device;

FIG. 8 illustrates two embodiments of the system of the disclosure; and

FIG. 9 illustrates the operational units of the system.

### DETAILED DESCRIPTION OF THE INVENTION

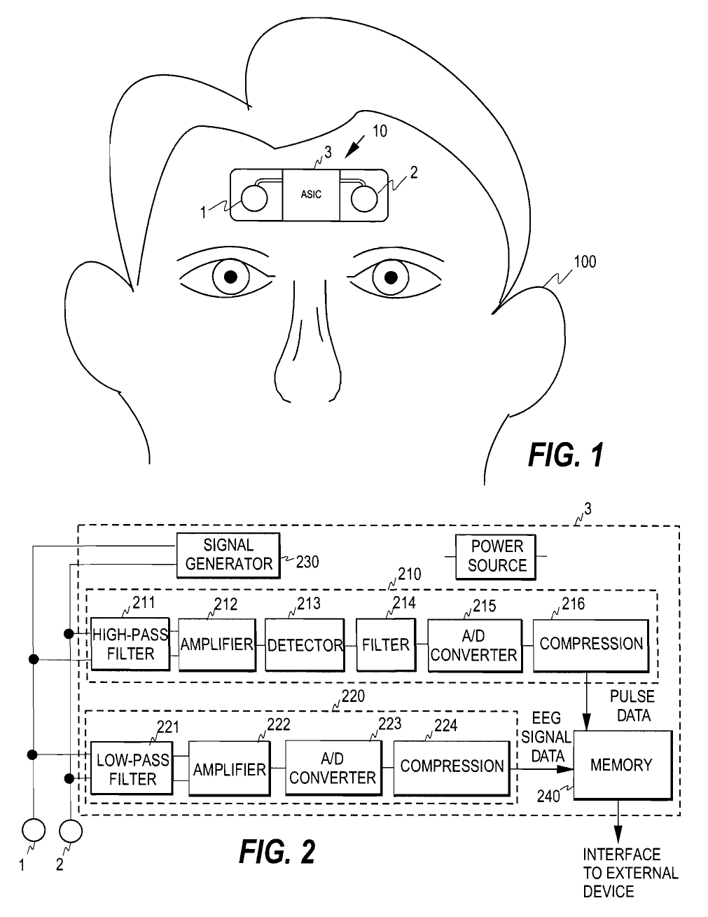

FIG. 1 illustrates a cordless recording device and a recording process for collecting physiological data from a patient. The recording device 10 is placed on the forehead of a patient 100 to collect biosignal data when the patient is sleeping. The patient may attach the recording device on his/her forehead in the evening and the device may collect data for a preset time, such as over the night. The recording device may then be detached and the data collected may be read from the memory of the device to perform a sleep analysis.

The recording device comprises a set of electrodes 1, 2 and an associated electronic circuitry 3, which may be in the form of an ASIC chip (Application Specific Integrated Circuit).

The mechanical structure of the cordless recording device 10 may be as is shown in the above-mentioned U.S. Patent Application US 2006/0007796. In other words, the electrodes, the electronic circuitry, including the memory, and the power source may be placed on a flexible substrate layer, such as a tape member and the recording device may be activated to start measuring when a protective layer covering the adhesive layer is removed right before attaching the device on the forehead.

In the embodiment of FIG. 1, the device is provided with two electrodes 1, 2 connected to the electronic circuitry with strip-like conductors. However, the number of the electrodes and their positions on the substrate layer may vary. In addition to EEG measurement electrodes, a neutral electrode may be used for balancing the potential of the instrument and the patient. Furthermore, the position of the ASIC chip with respect to the electrodes may vary. In the embodiment of FIG. 1, the chip is between the two electrodes to provide maximum distance between the electrodes. With respect to the general mechanical structure of the recording device, reference is made to the above-mentioned U.S. Patent Application US 2006/0007796.

FIG. 2 illustrates one embodiment of the electronic circuitry 3 of the cordless recording device. The core of the integrated digital data logger of the embodiment of FIG. 2 is a data memory 240 which stores two types of information measured through electrodes 1 and 2. First, brain wave signal data, typically EEG data, is collected into the data memory through a brain wave measurement branch 220. Second, pulse data is collected into the memory through an impedance measurement branch 210. Pulse data here refers to bioimpedance data including a pulsating component due to cardiac function or to any data which is refined from the said bioimpedance data and which is indicative of the HR of the patient.

An excitation signal of the bioimpedance measurement is fed to at least two electrodes, at least one of them being the same electrode from which the EEG signal is acquired. Specifically, the excitation signal may be fed to the neutral electrode.

For supplying the excitation signal in the embodiment of FIG. 1, the electronic circuitry includes a signal generator 230 connected to electrodes 1 and 2. The frequency of the excitation signal supplied to the patient through the electrodes is well above the EEG signal band, typically in the range of 20-100 kHz, in order to enable continuous and simultaneous bioimpedance measurement that does not interfere with the EEG measurement.

The carrier signal produced by the generator is measured from electrodes 1 and 2 by connecting the impedance measurement branch 210 to the electrodes. The impedance measurement branch includes a high-pass filter 211 at its front end. In case of more than two electrodes, the impedance measurement electrodes may be different than the excitation electrodes. Specifically, one measurement electrode may be the neutral electrode.

The low-pass filter 221 of the brain wave measurement branch 220 prevents high frequencies, i.e. the carrier signal, from entering the brain wave measurement branch, while the high-pass filter 211 prevents the low frequencies, i.e. the EEG signal, from entering the impedance measurement branch.

In the measurement branches the filtered signals are first amplified; the EEG signal is supplied to an amplifier 222 of the brain wave measurement branch, while the impedance signal is supplied to an amplifier 212 of the impedance measurement branch. The amplifiers are typically differential amplifiers.

The brain wave measurement branch further includes an A/D converter 223 that samples the EEG signal and converts it into digitized format. The A/D converter thus outputs a sequence of EEG signal data. After the low-pass filter 221, the EEG signal is processed in a conventional manner to obtain the said sequence. In addition, an optional compression unit 224 may precede the data memory to compress the EEG data before storing it.

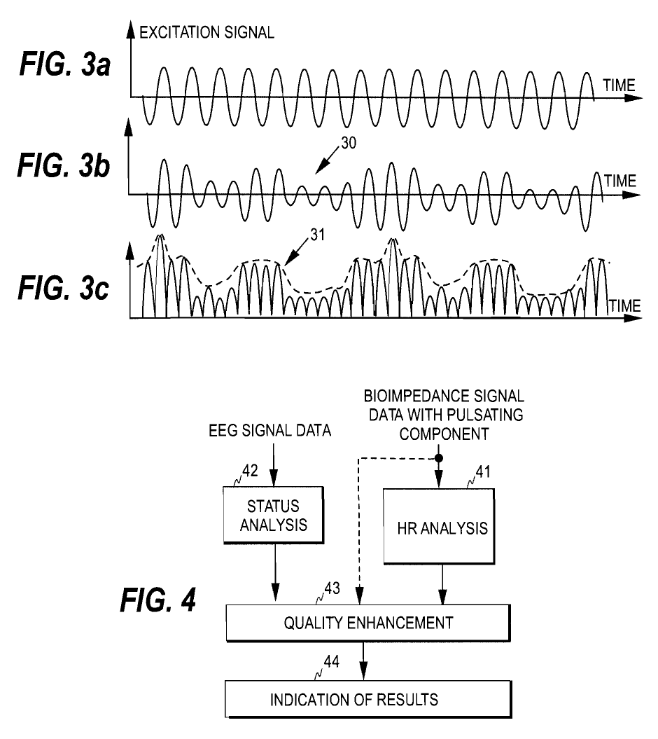

The signal generator 230 supplies an excitation current to the patient. The voltage between the electrodes, i.e. the amplitude of the carrier signal measured by the impedance measurement branch, is then proportional to the impedance of the signal path formed between electrodes 1 and 2. At this stage, the frequency content of the measured signal is concentrated around the frequency of the excitation current. FIGS. 3a and 3b illustrate, respectively, the excitation signal output from the signal generator and the carrier signal 30 output from amplifier 212. As can be seen, impedance changes cause slow changes in the amplitude of the signal.

The carrier signal is then demodulated in a detector 213 using the excitation frequency. This produces a time-varying signal indicating how the impedance of the signal path varies over time. As is shown in FIG. 3c, detector 213 typically outputs a bioimpedance signal 31, which corresponds to the envelope of the rectified input signal 30 and varies slowly over time in accordance with the impedance changes. A bioimpedance measurement similar to the above is disclosed in Applicant's European Patent Application EP 06 12 5862 where the bioimpedance signal is utilized to detect artifacts in EEG signal data.

In the present invention, the slowly-varying bioimpedance signal is filtered in a filter 214 to reduce the uninteresting frequency components of the signal. The filter is typically a band-pass filter that preserves the pulsating blood volume component of the bioimpedance signal, i.e. the passband includes the range of possible heart beat frequencies. The obtained bioimpedance signal, which is here termed pulse signal, is then converted into digitized format in an A/D converter 215. The A/D converter thus outputs a sequence of bioimpedance signal data that includes a pulsating component at the heart rate. An optional compression unit 216 may follow the A/D converter to compress the bioimpedance data before storing it into the data memory.

FIG. 4 is a flow diagram illustrating one embodiment of the overall analysis performed based on the bioimpedance and EEG data. The cordless recording device is provided with an interface through which the contents of the memory may be transferred to an external analysis device performing an off-line status analysis. Based on the collected bioimpedance signal including a pulsating component, a heart rate analysis is performed at step 41. The HR analysis includes generation of a HR time series. Based on the EEG signal data, a status analysis is performed at step 42 to obtain status information indicative of the status of the patient. The HR time series is used to enhance the quality of the measurement (step 43), by enhancing the information content of the status information provided to the user. Also the bioimpedance data may be utilized in the enhancement process, as is shown by the dashed arrow in FIG. 4. The status analysis may be a conventional sleep stage scoring process carried out based on EEG signal data. In this case, the results of the scoring process may presented to the user together with the heart beat characteristics temporally aligned with the sleep stage information, thereby to enhance the information content of the sleep stage information. A clinician may use the heart beat characteristics to evaluate why the status information is as it is. For example, sleep apnea patients have faster heart rates than non-apnea patients, but have less variability in their heart rates.

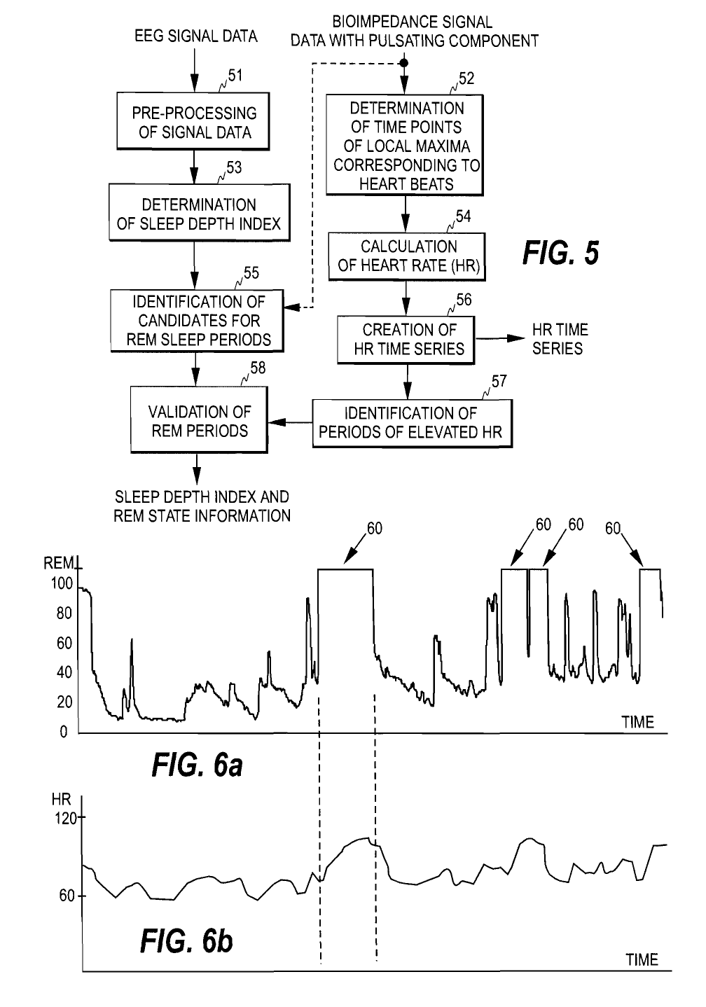

FIG. 5 illustrates one embodiment of the status and HR analyses carried out based on the bioimpedance and EEG data collected. The EEG signal data may first be pre-processed at step 51 to exclude undesirable components, such as artifacts, from the signal. The EEG signal data is then used to determine a time series of a sleep depth index at step 53. This may be carried out by continuously calculating the entropy values of the EEG signal data within successive and possibly overlapping time windows. The term entropy here refers to spectral entropy or to any other measure of the irregularity of the EEG signal data, such as Shannon entropy or approximate entropy. Based on the entropy sequence obtained, the process then determines sleep depth indices in successive time windows. The value of the index may be scaled to a fixed range, such as between 0 and 100, using an appropriate scaling function, for example. As a result, a time series of the sleep depth index is obtained from step 53. A method for determining a sleep depth index is disclosed in Applicant's U.S. patent application Ser. No. 11/438,637.

Candidates for REM sleep periods in the sleep depth index sequence are then detected (step 55). This may be carried out by based on electrooculographic (EOG) and/or electromyographic (EMG) data which may be extracted from the single EEG channel or obtained through a separate measurement channel. During REM periods, the EOG activity is high and EMG activity low, which allows the detection of REM period candidates.

In one embodiment of the invention, the detection of the REM sleep period candidates is performed by comparing the slowly varying bioimpedance signal with a predetermined threshold and determining the time periods during which the bioimpedance signal exceeds the threshold. These periods may be regarded as the REM sleep period candidates. Consequently, step 55 outputs the time series of the sleep depth index together with information about the possible periods of REM sleep. The pulsating component may be removed from the bioimpedance signal prior to the comparison.

The pulse data obtained from the data memory is processed to create a time series representing the heart rate of the patient. This may be carried out by first determining the time points of the local maxima corresponding to the heart beats (step 52). The heart rate may then be calculated, for example, by determining the average time difference between two consecutive maxima within a time window of a predetermined length, such as 5 seconds, and determining the inverse of the time difference (step 54). Consecutive HR values then form the HR time series (step 56). The time window may be sliding to obtain frequent update of the HR value.

The periods of elevated HR are then identified from the HR time series at step 57 and the validation of the REM periods is performed based on the elevated periods and the REM period candidates at step 58. The validation may be based on probabilities. The candidates for the REM periods may be associated with a first REM probability value at step 55 and the periods of elevated HR may be associated with a second REM probability value at step 57. At step 58, the periods for which both probabilities are high are selected as the REM periods. The sleep depth index and the REM periods are then displayed to the user as the result of the sleep analysis. This may be carried out by flagging the sleep depth index values obtained during the REM sleep periods or by replacing the index values obtained during REM sleep periods with new values indicative of a REM sleep state.

In further embodiment of the invention, the detection of the REM periods may also be based solely on either one of the bioimpedance and HR time series.

FIGS. 6a and 6b illustrate an example of the indication step 44. In this example, the sleep depth index is presented graphically on a scale from 0 to 100 and the validated REM periods 60 are shown as a separate REM state. Furthermore, as a result of the HR analysis, at least the HR time series may be presented graphically, as is shown in FIG. 6b. In addition to the HR time series, the HR variability may also be presented to the user to allow the user to evaluate why the sleep depth index acts as it does.

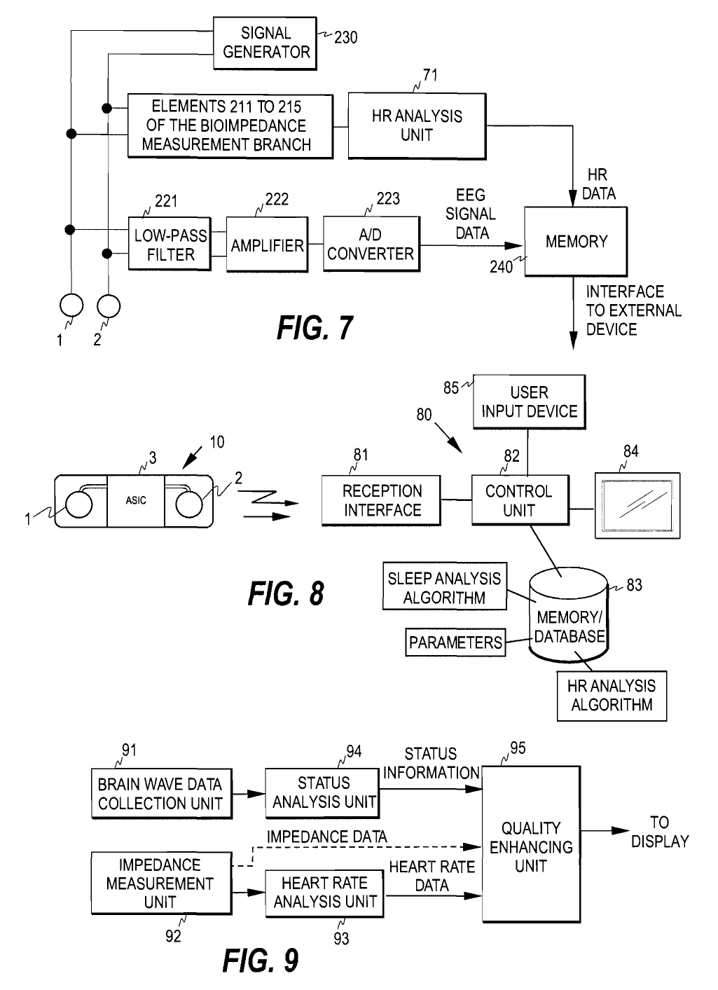

In the above embodiments of the invention, the bioimpedance data containing the pulsating component was stored in the memory of the recording device 10, and the HR analysis was performed off-line in an external analysis device after the recording of the data. However, the HR analysis or part of it may also be carried out in the recording device. FIG. 7 illustrates an example in which the recording device is configured to perform step 41 of FIG. 4 in an analysis unit 71. Thus, in this embodiment the recording device 10 stores the EEG signal data and HR time series. Otherwise the device of FIG. 7 corresponds to that of FIG. 2.

FIG. 8 illustrates one embodiment of the overall system of the invention. As discussed above, the operations of the invention may be distributed in various ways between the recording device 10 and the external analysis device 80, which may comprise any data processing unit/system provided with the necessary algorithms for utilizing the collected data and for displaying the results to the user of the device. Although the contents of the memory of the cordless recording device are typically uploaded through a standard interface, such as a USB port or a memory reader, the recording device and the external device may also be provided with a short-range transmitter-receiver pair for transferring the collected data wirelessly from the recording device to the external analysis device. Possible short-range wireless transmission technologies include Bluetooth, Wibree, and ZigBee, for example. It is also possible that the external analysis device analyzes the data transmitted wirelessly in an on-line manner. Therefore, the recording device does not necessarily comprise a data memory for storing the collected data, but at most a small buffer memory, if needed, for transmitting the collected data to the external analysis device substantially in an on-line manner according to the protocol of the wireless link.

The reception interface 81 of the external analysis device is typically connected to a control unit 82 provided with a memory or database 83 that may receive the collected data from the recording device. The memory or database of the external analysis device may also store the algorithms for analyzing the collected data, such as the algorithms for calculating the entropy values and the sleep depth indices and the HR analysis algorithms. Furthermore, the memory/database may include various parameters needed in the data processing, such as the threshold values with which the impedance signal and/or the HR values are compared.

Although a control unit comprising one computer or one processor unit may perform the above steps, the processing of the data may also be distributed among different units/processors (servers) within a network, such as a hospital LAN (local area network). The system of the invention may thus also be distributed between various processing units.

FIG. 9 illustrates the operational modules or units of the system. The system includes a brain wave data collection unit 91 configured to collect brain wave signal data from the subject through at least one electrode of the electrode set of the recording device and an impedance measurement unit 92 configured to measure an impedance signal through two electrodes of the set, the impedance signal being indicative of the bioimpedance of a signal path connecting the two electrodes. The system further includes a heart rate analysis unit 93 configured to derive heart rate data from the bioimpedance signal. As discussed above, the interface between the recording device and the external analysis device may vary so that the operations of the heart rate analysis unit may be entirely in either device or shared by the two devices. The system further includes a status analysis unit 94 configured to generate, based on the brain wave signal data, status information indicative of the status of the subject during the measurement period. The status information is supplied to a quality enhancing unit 95 configured enhance the quality of the status information based on the heart rate data available from the HR analysis unit. The quality enhancing unit may further utilize the impedance data available from the impedance measurement unit. As discussed above, both the status analysis unit and the quality enhancing unit are in the external analysis device. The operation of the quality enhancing unit may vary depending on how the quality of the status information is enhanced.

Although the present disclosure was described above with reference to the examples shown in the appended drawings, it is obvious that the disclosure is not limited to these, but may be modified by those skilled in the art without departing from the scope of the disclosure. For example, in addition to sleep analyses, the disclosure may be useful for studying daytime brain activity and vigilance of awake neurological patients.

We claim:

1. A method for analyzing the status of a subject, the method comprising the steps of:
providing a cordless recording device comprising a set of electrodes;
attaching the cordless recording device on the forehead of the subject;
collecting brain wave signal data from the subject through at least one electrode of the set;
measuring bioimpedance through two electrodes of the set, thereby to obtain an impedance signal indicative of the bioimpedance of a signal path connecting the two electrodes, wherein the collecting and the measuring are performed over a measurement period;
deriving heart rate data from the impedance signal, the heart rate data being indicative of the heart rate of the subject;
generating, based on the brain wave signal data, status information indicative of the status of the subject during the measurement period; and
utilizing the heart rate data to enhance the quality of the status information.

2. A method according to claim 1, further comprising storing the brain wave signal data into a data memory of the cordless recording device, wherein the generating and utilizing are performed off-line after the measurement period.

3. A method according to claim 1, wherein the generating includes performing a sleep analysis based on the brain wave signal data.

4. A method according to claim 2, further comprising storing the impedance signal in the data memory, wherein the deriving is performed off-line after the measurement period.

5. A method according to claim 2, further comprising storing the heart rate data in the data memory.

6. A method according to claim 3, wherein the performing includes generating a time series of a sleep depth index indicative of the depth of sleep of the subject and the utilizing includes producing information indicative of REM sleep periods in the time series.

7. A method according to claim 6, wherein the utilizing includes using the heart rate data to detect the REM sleep periods.

8. A method according to claim 1, wherein the utilizing includes presenting a time series of the heart rate data graphically to the user, the time series being temporally aligned with the status information.

9. A method according to claim 7, wherein the using includes detecting periods of elevated heart rate from the heart rate data.

10. An analysis system for analyzing the status of a subject, the system comprising:
a data collection unit configured to collect brain wave signal data from a subject through at least one electrode of a set of electrodes of a cordless recording device attachable to the forehead of the subject;
an impedance measurement unit configured to measure bioimpedance through two electrodes of the set, thereby to obtain an impedance signal indicative of the bioimpedance of a signal path connecting the two electrodes;
a heart rate analysis unit configured to derive heart rate data from the impedance signal, the heart rate data being indicative of the heart rate of the subject;
a status analysis unit configured to generate, based on the brain wave signal data, status information indicative of the status of the subject during the measurement period; and
a quality enhancing unit configured to enhance the quality of the status information based on the heart rate data.

11. An analysis system according to claim 10, wherein the status analysis unit is configured to perform a sleep analysis based on the brain wave signal data.

12. An analysis system according to claim 10, wherein the data collection unit and impedance measurement unit are in the cordless recording device and the heart rate analysis unit, the status analysis unit, and the quality enhancing unit are in an external analysis device.

13. An analysis system according to claim 10, wherein the data collection unit, the impedance measurement unit, and the heart rate analysis unit are in the cordless recording device and the status analysis unit and the quality enhancing unit are in an external analysis device.

14. An analysis system according to claim 10, wherein
the status analysis unit is configured to generate a time series of a sleep depth index indicative of the depth of sleep of the subject; and
the quality enhancing unit is configured to provide information indicative of REM sleep periods in the time series.

15. An analysis system according to claim 14, wherein the quality enhancing unit is configured to detect REM sleep periods based on the heart rate data.

16. An analysis system according to claim 10, wherein the quality enhancing unit is configured present the status information and a time series of the heart rate data graphically to the user, the time series being temporally aligned with the status information.

17. A cordless recording device for recording physiological data from a subject, the cordless recording device comprising:
a set of electrodes;
a first data collection unit configured to collect brain wave signal data from a subject through at least one electrode of the set;
an impedance measurement unit configured to measure bioimpedance through two electrodes of the set, thereby to obtain an impedance signal indicative of the bioimpedance of a signal path connecting the two electrodes; and
a second data collection unit configured to collect pulse data indicative of the heart rate of the subject.

18. A cordless recording device according to claim 17, further comprising a data memory operably connected to the set of electrodes, wherein the first data collection unit is further configured to store the brain wave signal data in the data memory and the second data collection unit is further configured to store the pulse data in the data memory.

19. A cordless recording device according to claim 17, wherein the second data collection unit is configured to derive the pulse data from the impedance signal.

20. A cordless recording device according to claim 18, wherein the second data collection unit is configured to store the impedance signal as the pulse data.

21. A cordless recording device according to claim 17, further comprising a short-range wireless transmitter configured to transfer the brain wave signal data and the pulse data to an external device.

22. An analysis system for analyzing the status of a subject, the system comprising:
data collection means for collecting brain wave signal data from a subject through at least one electrode of a set of electrodes of a cordless recording device attachable to the forehead of the subject;
impedance measurement means for measuring bioimpedance through two electrodes of the set, thereby to obtain an impedance signal indicative of the bioimpedance of a signal path connecting the two electrodes;
heart rate analysis means for deriving heart rate data from the impedance signal, the heart rate data being indicative of the heart rate of the subject;
status analysis means for generating, based on the brain wave signal data, status information indicative of the status of the subject during the measurement period; and
quality enhancing means for enhancing the quality of the status information based on the heart rate data.

23. A cordless recording device for recording physiological data from a subject, the cordless recording device comprising:
a set of electrodes;
first data collection means for collecting brain wave signal data from a subject through at least one electrode of the set;
impedance measurement means for measuring bioimpedance through two electrodes of the set, thereby to obtain an impedance signal indicative of the bioimpedance of a signal path connecting the two electrodes; and
second data collection means for collecting pulse data indicative of the heart rate of the subject.
